(19) United States
(12) Patent Application Publication
(10) Pub. No.: US 2008/0171095 A1
(43) Pub. Date: Jul. 17, 2008

(54) Carnosine-related compounds for preventing and treating impaired neurovascular blood flow

(75) Inventors: Arshad Majid, East Lansing, MI (US); Rajanikant Krisanamurthy, East Lansing, MI (US)

Correspondence Address: Peter G. Carroll;MEDLEN & CARROLL, LLP, Suite 350, 101 Howard Street, San Francisco, CA, 94105, US

(73) Assignee: Michigan State University

(21) Appl. No.: 11/441,376

(22) Filed: May 25, 2006

Related U.S. Application Data
(60) Provisional application No. 60/684,288, filed on May 25, 2005.

(51) Int. Cl. A61K 33/34; A61K 38/05; A61K 33/30; A61K 33/26; A61K 33/06; A61K 33/32

(52) U.S. Cl. 424/638; 514/19; 424/639; 424/646; 424/641; 424/682

(57) ABSTRACT

The present invention discloses a dipeptide that is useful for the prevention or treatment of impaired neurovascular blood flow. The invention teaches that administration of carnosine-related compounds reduce focal ischemia (i.e., for example, stroke) symptomology whether administered before, or after, the impaired blood flow.

### FIELD OF INVENTION

The present invention relates to treatment of neurovascular disease. In one embodiment, the present invention relates to the treatment of impaired neurological blood flow. In one embodiment, the impaired neurological blood flow comprises stroke. In one embodiment, a dipeptide reduces symptoms of stroke. In another embodiment, the dipeptide is carnosine.

### BACKGROUND

Free radicals and the peroxidative processes caused by free radicals have been known for a long time to be one of the causes of the structural and functional degradations of bodily tissues and are responsible for a number of pathologies. Many pathologies involving oxidative tissue damage occur in the neurovasculature. One particular pathology related to oxidative tissue damage is stroke, even though the exact mechanism of action is unknown.

Antioxidant compounds are believed important in protecting the body from scavenging “free radicals” that oxidize body tissue under a number of circumstances. Studies recommending antioxidants as prophylactic or actual treatments for pathological conditions involving free radical generation are general in nature and treat the patient from a “whole body” concept (i.e., for example, homeopathy).

What is needed in the art are specific antioxidant compounds that can prevent and/or treat significant neurovascular disorders, such as stroke.

### SUMMARY

The present invention relates to treatment of neurovascular disease. In one embodiment, the present invention relates to the treatment of impaired neurological blood flow. In one embodiment, the impaired neurological blood flow comprises stroke. In one embodiment, a dipeptide reduces symptoms of stroke. In another embodiment, the dipeptide is carnosine.

In one embodiment the present invention contemplates a method, comprising: a) providing; i) a patient having at least one symptom of an impaired neurovascular blood flow; ii) a composition comprising a carnosine-related (e.g., carnosine or a derivative thereof) compound; and b) administering said composition to said patient under conditions such that at least one symptom of said impaired neurovascular blood flow is reduced. In one embodiment, said impaired neurovascular blood flow comprises a stroke. In one embodiment, said symptom is selected from the group consisting of i) weakness or paralysis of an arm, leg, side of the face, or any part of the body; ii) numbness, tingling, decreased sensation; iii) vision changes; iv) slurred speech; v) inability to speak or understand speech; vi) difficulty reading or writing; vii) swallowing difficulties or drooling; viii) loss of memory; ix) vertigo; x) loss of balance or coordination; xi) personality changes; xii) mood changes; xiii) drowsiness, lethargy, or loss of consciousness; and xiv) uncontrollable eye movements or eyelid drooping. In one embodiment, said carnosine-related compound is selected from the group consisting of carnosine, homocarnosine, anserine, and ofidine. In one embodiment, said composition further comprises a branched amino acid. In one embodiment, said composition further comprises carnitine. In one embodiment, said composition further comprises creatine. In one embodiment, said composition further comprises an element, wherein said element is selected from the group consisting of iron, zinc, manganese, magnesium, copper, cobalt, chrome, molybdenum, vanadium and selenium. In one embodiment, said composition further comprises a vitamin wherein said vitamin is selected from the group consisting of Vitamin A, Vitamin B15, Vitamin C, Vitamin D3, and Vitamin E. In one embodiment, said composition further comprises a bioflavonoid. In one embodiment, said patient is human.

In one embodiment, the present invention contemplates a method, comprising: a) providing; i) a patient comprising at least one risk factor for an impaired neurovascular blood flow; ii) a composition comprising a carnosine-related (e.g., carnosine or a derivative thereof) compound; and b) administering said composition to said patient under conditions such that said impaired neurovascular blood flow is avoided. In one embodiment, said impaired neurovascular blood flow comprises a stroke. In one embodiment, said risk factors are selected from the group consisting of high blood pressure, age, family history, smoking, diabetes, high cholesterol, heart disease, birth control pill use, pregnancy, cocaine use, alcohol abuse, head injury, and bleeding disorders. In one embodiment, said carnosine-related compound is selected from the group consisting of carnosine, homocarnosine, anserine, and ofidine. In one embodiment, said composition further comprises a branched amino acid. In one embodiment, said composition further comprises carnitine. In one embodiment, said composition further comprises creatine. In one embodiment, said composition further comprises an element, wherein said element is selected from the group consisting of iron, zinc, manganese, magnesium, copper, cobalt, chrome, molybdenum, vanadium and selenium. In one embodiment, said composition further comprises a vitamin wherein said vitamin is selected from the group consisting of Vitamin A, Vitamin B15, Vitamin C, Vitamin D3, and Vitamin E. In one embodiment, said composition further comprises a bioflavonoid. In one embodiment, said patient is human.

### DEFINITIONS

The terms used in describing the present invention should be interpreted as commonly accepted by those having skill in the art, with the notable exceptions of those listed below.

The term “stroke” as used herein, means an interruption of the blood supply (i.e., for example, impaired neurovascular blood flow) to any part of the brain, resulting in damaged brain tissue. Symptoms of stroke depend on what part of the brain is damaged. In some cases, a patient (i.e., for example a human) may not even be aware that he or she has had a stroke. Usually symptoms include, but are not limited to: i) weakness or paralysis of an arm, leg, side of the face, or any part of the body; ii) numbness, tingling, decreased sensation; iii) vision changes; iv) slurred speech; v) inability to speak or understand speech; vi) difficulty reading or writing; vii) swallowing difficulties or drooling; viii) loss of memory; ix) vertigo (spinning sensation); x) loss of balance or coordination; xi) personality changes; xii) mood changes (depression, apathy); xiii) drowsiness, lethargy, or loss of consciousness; and ivx) uncontrollable eye movements or eyelid drooping.

The term “patient”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

### BRIEF DESCRIPTION OF THE DRAWINGS

FIG. 1 presents exemplary data showing the effects of pre-occlusion intraperitoneal (IP) administration of carnosine on focal ischemia. Open Bar: Saline. Vertically Striped Bar: 100 mg/kg carnosine. Crosshatched Bar: 500 mg/kg carnosine. Vertical Axis: Infarct volume (mm³).

FIG. 2 presents exemplary data showing the effects of pre-occlusion and post-occlusion intraperitoneal (IP) administration of carnosine on focal ischemnia. Open Bar: Saline. Vertically Striped Bar: Pre-occlusion 100 mg/kg carnosine. Crosshatched Bar: Pre-occlusion 500 mg/kg carnosine. Horizontally Striped Bar: Post-occlusion 1000 mg/kg. Vertical Axis: Infarct volume (mm³).

### DETAILED DESCRIPTION OF THE INVENTION

The present invention relates to treatment of neurovascular disease. In one embodiment, the present invention relates to the treatment of impaired neurological blood flow. In one embodiment, the impaired neurological blood flow comprises stroke. In one embodiment, a dipeptide reduces symptoms of stroke. In another embodiment, the dipeptide is carnosine.

### Stroke

Stroke, or brain attack as it is commonly called, can be caused by either vascular hemorrhage or vascular blockage with the latter accounting for about 80% of the events which lead to a stroke. Vascular hemorrhage is also term-ed a hemorrhagic stroke or an aneurism. Vascular blockage may also be termed ischemic stroke. Both types of stroke are associated with considerable morbidity in terms of long-term neurological deficit and the risk of subsequent stroke as well as mortality post stroke.

Stroke accounts for 1 out of every 15 deaths in the United States. It is the 3rd leading cause of death in most developed countries, and the leading cause of disability in adults. The risk doubles with each decade after age 35. If the flow of blood in an artery supplying the brain is interrupted for longer than a few seconds, brain cells can die, causing permanent damage. An interruption can be caused by either blood clots or bleeding in the brain. Most ischemic strokes are due to blood clots that block blood flow.

Hemoragghic strokes are due to bleeding into the brain subsequent to a blood vessel rupture or significant injury.

One cause of ischemic stroke is atherosclerosis. Fatty deposits and blood platelets collect on the wall of the arteries, forming plaques. Over time, the plaques slowly begin to block the flow of blood. The plaque itself may block the artery enough to cause a stroke. Often, the plaque causes the blood to flow abnormally, which leads to a blood clot. A blood clot can stay at the site of vascular narrowing (i.e., thereby becoming a thrombus) and prevent blood flow to all of the smaller arteries it supplies. In other cases, the blood clot can travel (i.e., thereby becoming an embolism) and wedge into a smaller vessel.

Ischemic strokes caused by embolism are most commonly caused by heart disorders. An embolism may originate in a major blood vessel as it branches off the heart. A blood clot can also form elsewhere in the body for any number of reasons, and then travel to the brain, causing a stroke.

Arrhythmias of the heart, such as atrial fibrillation, can be associated with ischemic stroke and may contribute to blood clot formation. Other causes of embolic ischemic stroke include endocarditis (an infection of the heart valves), or use of a mechanical heart valve. A blood clot can form on the artificial valve, break off, and travel to the brain.

A hemorrhagic stroke may occur when small blood vessels in the brain become weak and burst. Some people have defects in the blood vessels of the brain that make this more likely than in other people. The flow of blood after the blood vessel ruptures damages brain cells.

High blood pressure is the number one causative risk factor in most strokes. The risk of stroke is also increased by age, family history of stroke, smoking, diabetes, high cholesterol, and heart disease. Certain medications promote blood clot formation and may increase chances for a stroke (i.e., for example, birth control pill use, especially if a woman taking them also smokes and is older than 35). Women also have a higher risk of stroke during pregnancy and the weeks immediately after pregnancy. Overall, however, more men have strokes than women. Cocaine use, alcohol abuse, head injury, and bleeding disorders increase the risk of bleeding into the brain (i.e., for example, a hemoragghic stroke).

Stroke treatment in the acute phase typically entails the invasive administration of blood clot dissolving drugs within the first three hours of the stroke as well as stabilization of cardiovascular functions and vital signs. After treatment in the acute phase, patients may typically follow four pathways: (i) in the case of mild stroke the patient may go home, (ii) in the case of a more severe stroke where it is believed an improvement in outcome can occur the patient may be sent to rehabilitation, (iii) other patients may be sent to special care/nursing home, and (iv) some patients die.

In certain embodiments, a patient can be a human that has been identified as being susceptible to ischemia, including stroke, using any available method, including, but not limited to, diagnostic procedures using: computed tomography (CT), magnetic resonance imaging (MRI, including DWI and PWI), carotide ultrasonography/doppler scanning, magnetic resonance angiography (MRA), carotid angiography, chest X-ray, electrocardiography (ECG, or EKG), echocardiography, Holter monitoring or telemetry, and the like.

While the use of a blood thinning agents such as Tissue Plasminogen Activator or coumadin (warfarin) may be used in the treatment of ischemic stroke, it may be the wrong therapy to give in the case of a hemorrhagic stroke. Hence it is important to determine the type of stroke which has occurred in the patient. Furthermore, the drug therapy may vary during the course of treatment depending on the progress which the patient makes. Hence it is important to develop effective treatments that have widespread patient compatibility.

Many experimental models are reported by those having skill in the art in an attempt to study stroke. However, stroke models imparting a general ischemic condition do not replicate the conditions that generally occur during a typical stroke. The protective effect of carnosine to generalized ischemia was evaluated in studies using generalized hypoxia, acoustic stress, and bilateral carotid artery occlusion Stovlinsky et al. “Anti-Ischemic Activity Of Carnosine” Biochiemistry (Moscow) 65:849-855 (2000). A typical stroke usually occurs as a localized (i.e., focal) event. Consequently, experimental models that create focal ischemia are considered most relevant to the occurrence of stroke.

For example, focal ischemia may be created by a temporary three-vessel occlusion (3VO) technique. This technique uses a surgical approach which blockes (i.e., occludes) the middle cerebral artery (MCA) and produces a consistent cerebral infarction volume. An intraluminal thread-occlusion technique (performed by using endoscopy) targeting the MCA occlusion (MCAO) is more widely used since it does not require complicated intracranial procedures. Other methods and models for MCAO stroke are also known that provide consistent degrees and variance of cortical stroke injury. One model uses a modified temporary 3VO technique and requires less complicated procedures than the temporary 3VO model, i.e., temporary occlusion of the bilateral common carotid arteries (CCAs) superimposed on a permanent occlusion of the MCA. In microvascular cerebral perfusions, significant reductions in regional cerebral perfusion during the 3VO protocol was followed by a rapid return to baseline after release of the CCAs, showing that the technique induces a temporary focal ischemia. The average sizes and variances of the neocortical infarction in this model, together with those in the other normotensive rat models caused by the 3VO technique in the literature, indicated a standard size and variance of infarcted lesion in the control groups relative to the specific ischemic period. However, stroke injuries in the neocortex induced by the thread occlusion technique showed greater variability with less consistent lesion sizes. As an added complication, inclusion/exclusion criteria to avoid ischemic injury severity that is either too mild (i.e., no and/or faint infarction) or too great (i.e., huge and/or fatal infarction) severity differ between laboratories in the thread occlusion model. Yanamoto et al., “Evaluation Of MCAO Stroke Models In Normotensive Rats: Standardized Neocortical Infarction By The 3VO Technique” Exp Neurol. 182(2):261-74 (2003).

Nitroxide antioxidants are reported to have the ability to protect against focal ischemia. Maxwell K., “Prophylactic Pretreatment With Antioxidants” WO 04/096219 (2003). Maxwell does not teach that carnosine, alone, protects against focal ischemia. A combination treatment, however, is suggested using nitroxides with a laundry-list of other free-radical scavenging antioxidants that, by chance, includes carnosine. Other investigators have made invitations to try treating generalized ischemic conditions (or any other disease included in a laundry list that, by chance, includes stroke) by creating a combination treatment with a specific preferred compound with a second laundry list of antioxidant compounds (i.e., for example, cystine derivatives with other antioxidants that, by chance, includes carnosine). Nakano el al., “Novel Cysteine Derivative And Agent For Suppressing Activation Of Inflammatory Factors”, United States Patent Publication No. 2004/0059110. Filed: Aug. 4, 2003. The present invention contemplates embodiments that have specifically provided explicit teachings that carnosine-related compounds by themselves, when administered properly, both protect against and treat stroke symptomology.

### Carnosine

Carnosine is a naturally occurring dipeptide found within glia and neurons of the brain that exhibits features characteristic of a neurotransmitter. Carnosine modulates the effect of zinc and copper released at synapses during neuronal activity, which have been linked to damage associated with Alzheimer's disease, stroke, and seizures. Tombley et al., “Interactions Between Carnosine And Zinc And Copper: Implications For Neuromodulation And Neuroprotection” Biochemistry (Moscow) 65:807-816 (2000); and Horning et al., “Endogenous Mechanisms Of Neuroprotection: Role Of Zinc, Copper, And Carnosine” Brain Res 852:56-61 (2000). Carnosine also has antioxidant properties, and therefore may be useful for the prevention or treatment of oxidative damage in a number of neurological diseases, including stroke. Stvolinsky et al., “Carnosine: An Endogenous Neuroprotector In The Ischemic Brain” Cell Mol Neurobiol 19:45-56 (1999).

Stroke is one of the leading causes of death and disability in the United States. Carnosine has previously been shown to provide protection against ischemia in cultured cells and global ischemia in rats. Trombley et al., supra. In one embodiment, the present invention contemplates that carnosine is an effective compound to either prevent, or treat, localized (i.e., focal) ischemia. In one embodiment, a focal ischemia comprises stroke.

Carnosine (β-alanyl-L-histidine) and carnosine derivatives (i.e., for example, homocarnosine, acetylcarnosine, acetylhomocarnosine, etc.) have been known for some time to be among the most important natural antioxidant agents. Boldyrev et al., (1990), Adv. Enz. Reg., 30. 175-194; Kohen R. et al., (1988), Proc. Natl. Acad. Sci. USA, 85. 3175-79; Yoshikawa et al., (1991), Biochim. Biophys. Acta, 1115. 15-22. Consequently, it was speculated that the administration of carnosine and carnosine derivatives might provide a potential therapy for a number of pathologies. Davey C. L., (1960), Arch. Biochem. Biophys., 89. 303-308; Severin S., (1964); Proc. 6th Intern. Biochem. Congress, 45-61; Nagai et al., (1988), Meth. Find. Exp. Clin. Pharmacol., 10. 497-507; Boldyrev A., (1990), Int. J. Biochem., 22. 129-132; Kurelle et al., (1991), Byul. Exp. Biol. Med., 112. 52-53; Boldyrev et al.; (1993), Int. J. Biochem., 25 1101-1107; Boldyrev et al., (1993), Mol. Chem. Neuropathol., 19. 185-192. These studies focused on conditions where the peroxidative damage induced by free radicals is one of the main causes in inducing and/or worsening tissue damage. Although it is not necessary to understand the mechanism of an invention, it is believed that the antioxidant activity of exogenous carnosine (or carnosine-related compounds such as carnosine derivatives) might be restricted by a degradative enzyme (carnosinase) which is capable of hydrolyzing carnosine into amino acid components (i.e., alanine and histidine).

### Compositions

The present invention contemplates embodiments comprising a composition that contains carnosine-related (e.g., carnosine and derivatives thereof) compounds including, but not limited to, carnosine (β-alanyl-L-histidine), homocarnosine (τ-butyryl-L-histidine), anserine (N₁-methyl-β-alanyl-L-histidine), ofidine (N₃-methyl-β-alanyl-L-histidine), and/or the pharmacologically compatible inorganic or organic salts thereof and/or the acyl derivatives thereof with pharmacologically compatible acids organic, and the inorganic salts thereof. In one embodiment, the carnosine-related compounds in the composition ranges from approximately 1% to 50% by weight (with the exception of the inert material used for the galenical formulation, also including any sugars added and flavors), but more preferably from 15% to 25% by weight.

In one embodiment, the composition may further comprise one or more branched amino acids, in suitable ratios, such as, but not limited to: leucine, isoleucine and valine, (either in free form or as basic or acid inorganic/organic salts), provided that they are pharmacologically compatible, and/or the acyl derivatives thereof, salified with inorganic or organic bases, and/or the esters thereof with straight or branched alcohols, optionally salified with inorganic or organic acids. The weight ratio of leucine, isoleucine and valine may range from approximately 1:1:1 to 1:0:0, wherein compositions in which leucine is at least 20% of the mixture of the three amino acids being preferred. In one embodiment, the amino acids in the composition ranges from approximately 5% to 80% by weight (with the exception of the inert materials used for the galenical formulation, also including any sugars added and flavors), ranging preferably from 50% to 70%.

In one embodiment, the composition may further comprise carnitine (3-carboxy-2-hydroxy-N,N,N-trimethyl-1-propanamine inner salt) and/or water-soluble acyl derivatives thereof (i.e., for example, acetylcarnitine, propionylcarnitine, etc.), or creatine. In one embodiment, the carnitine or creatine ranges from approximately 0% to 20% by weight (with the exception of the inert materials used for the galenical formulation, also including any sugars added and flavors), but more preferably from 2.5% to 5% by weight.

In one embodiment, the composition may further comprise trace elements such as, but not limited to, iron, zinc, manganese, magnesium, copper, cobalt, chrome, molybdenum, vanadium and selenium in the form of salts (fumarate, sulfate, oxide, etc.) In one embodiment, the trace elements in the composition ranges from approximately 0 mg to 30 mg, depending on the trace element in the final galenical formulation.

In one embodiment, the composition may form complexes with any pharmacologically compatible amino acids, polypeptides or proteins.

In one embodiment, the composition further comprises vitamins including, but not limited to Vitamin A, Vitamin B15, Vitamin C, Vitamin D3, or Vitamin E in amounts generally recommended for nutritional supplementation.

In another embodiment, the composition further comprises bioflavonoids including, but not limited to, those derived from citrus fruits (i.e., for example, orange, lemon, grapefruit) in amounts generally recommended for nutritional supplementation.

### Pharmaceutical Formulations

Carnosine and its pharmaceutically acceptable derivatives to be used as antioxidant agents can be prepared easily using pharmaceutical materials which themselves are available in the art and can be prepared by established procedures.

The preparation of pharmaceutical formulations of the composition contemplated by present invention for oral administration need no specific techniques, since the different powders have a good mixibility and/or are easily water-soluble also in admixture. Alternatively, formulations may be made to accommodate composition administration by other routes including, but not limited to, intraperitoneal injection, intravenous injection, intramuscular injection, parenteral, intranasal, sublingual, inhalation (i.e., for example, by an aerosol), suppository, etc. The appropriate solutions and formulations for these routes of administration are known in the art.

The compositions of the present invention may be prepared using traditional oral pharmaceutical forms: tablets, divisible or not; suitably flavored chewable tablets; hard- and soft-gelatin capsules; granulates for the extemporary preparation of aqueous solutions, suitably flavored and added with pharmacologically inert excipients such as various sugars (sachets, solids for use in plunger caps, etc.); suitably flavored chewing gums; wafer sheets; ready-to-use aqueous solutions, optionally flavored and added with suitable stabilizers, etc.

The compositions can also be formulated using capsules or soluble, effervescent tablets, or sachets, etc., after preparing a humid granulate in which the branched amino acids are dissolved in water and the solution is subsequently sprayed on a homogeneous solid mixture obtained by dry mixing (i.e., for example, in suitable coating pans) of the other suitably powdered components (i.e., for example, in a ball mill). In one embodiment, the final mixture is then dried in dry air stream at a temperature below 45° C.

In one embodiment, a soluble effervescent formulation may be prepared using a dry tartaric or citric acid that are added in a controlled-humidity environment and mixed to homogeneity. Optionally, the procedure may be repeated with sodium bicarbonate. After that, the product can be granulated with techniques known to those having skill in the art. In one embodiment, the compression of a final mixture creates effervescent tablets or sachets. Alternatively, a direct partition of the above granulate results in the preparation of capsules or tablets (i.e, without the added effervescent components).

Table 1 presents examples of formulations comprising embodiments of the compositions contemplated by the present invention. The active ingredient contents are expressed in grams, independently of the salification, based upon 100 g of composition.

TABLE 1
Representative Carnosine Compositions

| Nomenclature | Composition Element | Amount in grams |
|---|---|---|
| Formulation I | Carnosine (base or | 15 |
| | hydrochloride) | |
| | Leucine (base, hydrochloride, | 15 |
| | sulfate, acetate etc.) | |
| | Isoleucine (base, hydrochloride, | 15 |
| | sulfate, acetate etc.) | |
| | Valine (base, hydrochloride, | 15 |
| | sulfate, acetate etc.) | |
| | Carnitine (base, hydrochloride | 15 |
| | etc.) | |
| | Arginine (base, hydrochloride, | 15 |
| | glutamate etc.) | |
| | Mg (carbonate-hydroxide, basic | 8.5 |
| | citrate, lactate, sulfate) | |
| | Zinc (carbonate, lactate, sulfate | 0.5 |
| | etc.) | |
| | Copper (acetate, basic carbonate, | 0.25 |
| | gluconate, sulfate etc.) | |
| | Iron (gluconate, albuminate, | 0.75 |
| | fumarate, proteinate etc.) | |
| Formulation II | Carnosine (base or | 35 |
| | hydrochloride) | |
| | Leucine (base, hydrochloride, | 50 |
| | sulfate, acetate etc.) | |
| | Arginine (base, hydrochloride, | 10 |
| | glutamate etc.) | |
| | Mg (carbonate-hydroxide, basic | 3.5 |
| | citrate, lactate, sulfate) | |
| | Zinc (carbonate, lactate, sulfate | 0.5 |
| | etc.) | |
| | Copper (acetate, basic carbonate, | 0.25 |
| | gluconate, sulfate etc.) | |
| | Iron (gluconate, albuminate, | 0.75 |
| | fumarate, proteinate etc.) | |
| Formulation III | Carnosine (base or | 18 |
| | hydrochloride) | |
| | Leucine (base, hydrochloride, | 16 |
| | sulfate, acetate etc.) | |
| | Isoleucine (base, hydrochloride, | 16 |
| | sulfate, acetate etc.) | |
| | Valine (base, hydrochloride, | 16 |
| | sulfate, acetate etc.) | |
| | Carnitine (base, hydrochloride | 7.5 |
| | etc.) | |
| | Creatine (base) | 7.5 |
| | Arginine (base, hydrochloride, | 15 |
| | glutamate etc.) | |
| | Mg (carbonate-hydroxide, basic | 2.5 |
| | citrate, lactate, sulfate) | |
| | Zinc (carbonate, lactate, sulfate | 0.5 |
| | etc.) | |
| | Copper (acetate, basic carbonate, | 0.25 |
| | gluconate, sulfate etc.) | |
| | Iron (gluconate, albuminate, | 0.75 |
| | fumarate, proteinate etc.) | |
| Formulation IV | Carnosine (base or | 25 |
| | hydrochloride) | |
| | Leucine (base, hydrochloride, | 25 |
| | sulfate, acetate etc.) | |
| | Isoleucine (base, hydrochloride, | 10 |
| | sulfate, acetate etc.) | |
| | Valine | 6 |
| | Creatine (base) | 20 |
| | Arginine (base, hydrochloride, | 10 |
| | glutamate etc.) | |
| | Mg (carbonate-hydroxide, basic | 2.5 |
| | citrate, lactate, sulfate) | |
| | Zinc (carbonate, lactate, sulfate | 0.5 |
| | etc.) | |
| | Copper (acetate, basic carbonate, | 0.25 |
| | gluconate, sulfate etc.) | |
| | Iron (gluconate, albuminate, | 0.75 |
| | fumarate, proteinate etc.) | |
| Formulation V | Carnosine (base or | 20 |
| | hydrochloride) | |
| | Leucine (base, hydrochloride, | 25 |
| | sulfate, acetate etc.) | |
| | Isoleucine (base, hydrochloride, | 15 |
| | sulfate, acetate etc.) | |
| | Creatine (base) | 20 |
| | Arginine (base, hydrochloride, | 15 |
| | glutamate etc.) | |
| | Mg (carbonate-hydroxide, basic | 2.5 |
| | citrate, lactate, sulfate) | |
| | Zinc (carbonate, lactate, sulfate | 0.5 |
| | etc.) | |
| | Copper (acetate, basic carbonate, | 0.15 |
| | gluconate, sulfate etc.) | |
| | Iron (gluconate, albuminate, | 0.75 |
| | fumarate, proteinate etc.) | |
| | Manganese (sulfate, gluconate) | 0.10 |
| | Vitamins B15/C/E/D3 | 150 mg |
| | Lemon and orange bioflavonoids | 200 mg |

### Experimental

The following examples are merely illustrative of one embodiment of the present invention and are not to be construed as a limitation thereof.

### EXAMPLE 1

### Reduction of Focal Ischemia in Mice

This example provides illustrative data showing that carnosine administration protects against focal ischemia (i.e., stroke) in mice.

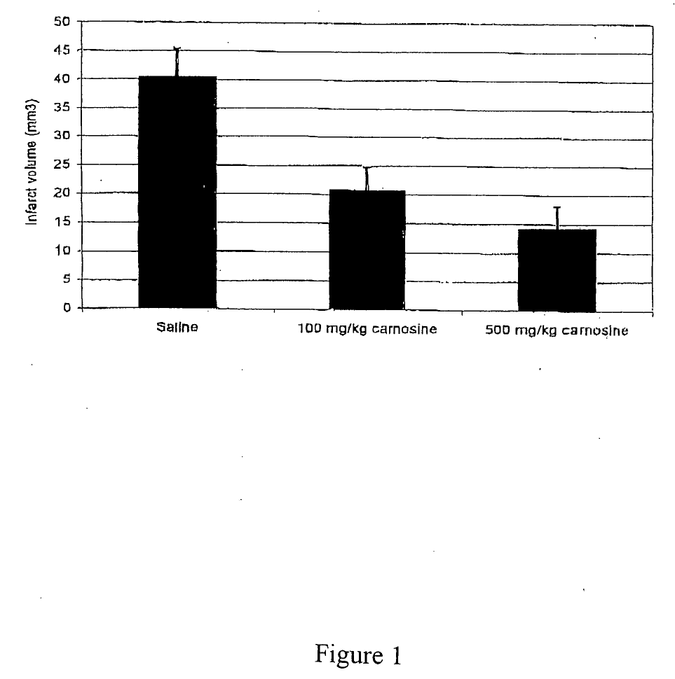

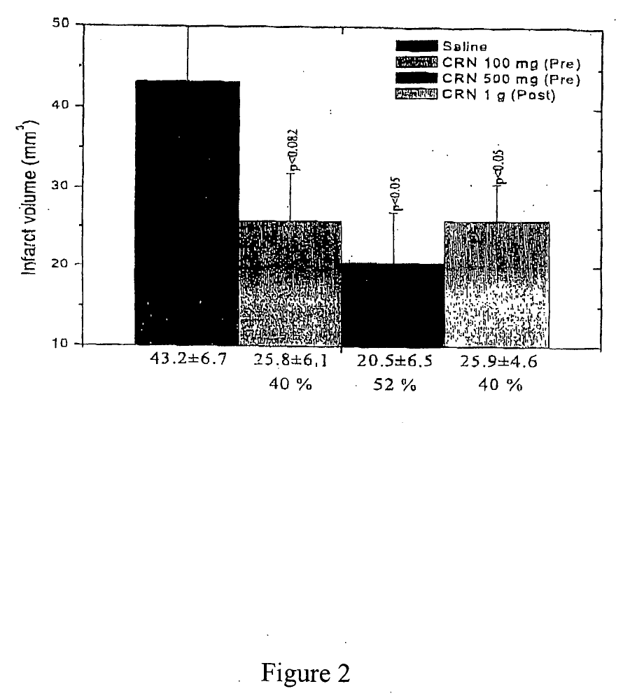

Male C57BL/6J mice were subjected to a permanent occlusion of the middle cerebral artery. For prophylactic studies, carnosine (100 mg/kg or 500 mg/kg, I.P.) was administered thirty (30) minutes (FIG. 1) or sixty (60) minutes (FIG. 2) before artery occlusion. For treatment studies, carnosine (1000 mg/kg, I.P.) was administered sixty (60) minutes after artery occlusion (FIG. 2).

Damage to the neural tissue (i.e., infarct volume) was assessed twenty-four (24) hours after occlusion by 2,3,5-triphenyl-tetrazolium chloride staining. Infarct volumes were reduced by 49% and 65% in mice prophylactically given 100 mg/kg and 500 mg/kg carnosine, respectively, versus saline-injected control mice. (FIG. 1) This protective dose dependence was reproduced in a second experiment that compared a post-occlusion carnosine administration. In this experiment, infarct volumes were reduced by 40% and 52%, respectively in mice prophylactically given 100 mg/kg and 500 mg/kg carnosine. A 40% reduction in infarct volume was also observed when mice were administered 1000 mg/kg carnosine after the occlusion.

These results show that carnosine protects against the development of focal ischemia (i.e., stroke) and provides an effective treatment of an actual stroke event.

1. A method, comprising:
a) providing;
i) a patient having at least one symptom of an impaired neurovascular blood flow;
ii) a composition comprising a compound, wherein said compound is selected from the group consisting of carnosine and a derivative thereof; and
b) administering said composition to said patient under conditions such that at least one symptom of said impaired neurovascular blood flow is reduced.

2. The method of claim 1, wherein said symptom is selected from the group consisting of i) weakness or paralysis of an arm, leg, side of the face, or any part of the body; ii) numbness, tingling, decreased sensation; iii) vision changes; iv) slurred speech; v) inability to speak or understand speech; vi) difficulty reading or writing; vii) swallowing difficulties or drooling; viii) loss of memory; ix) vertigo; x) loss of balance or coordination; xi) personality changes; xii) mood changes; xiii) drowsiness, lethargy, or loss of consciousness; and xiv) uncontrollable eye movements or eyelid drooping.

3. The method of claim 1, wherein said compound is selected from the group consisting of carnosine, homocarnosine, anserine, and ofidine.

4. The method of claim 1, wherein said composition further comprises a branched amino acid.

5. The method of claim 1, wherein said composition further comprises carnitine.

6. The method of claim 1, wherein said composition further comprises creatine.

7. The method of claim 1, wherein said composition further comprises an element, wherein said element is selected from the group consisting of iron, zinc, manganese, magnesium, copper, cobalt, chrome, molybdenum, vanadium and selenium.

8. The method of claim 1, wherein said composition further comprises a vitamin wherein said vitamin is selected from the group consisting of Vitamin A, Vitamin B15, Vitamin C, Vitamin D3, and Vitamin E.

9. The method of claim 1, wherein said composition further comprises a bioflavonoid.

10. The method of claim 1, wherein said patient is human.

11. A method, comprising:
a) providing;
i) a patient comprising at least one risk factor for an impaired neurovascular blood flow;
ii) a composition comprising a compound, wherein said compound is selected from the group consisting of carnosine and a derivative thereof; and
b) administering said composition to said patient under conditions such that said impaired neurovascular blood flow is avoided.

12. The method of claim 1l, wherein said risk factors are selected from the group consisting of high blood pressure, age, family history, smoking, diabetes, high cholesterol, heart disease, birth control pill use, pregnancy, cocaine use, alcohol abuse, head injury, and bleeding disorders.

13. The method of claim 11, wherein said compound is selected from the group consisting of carnosine, homocarnosine, anserine, and ofidine.

14. The method of claim 11, wherein said composition further comprises a branched amino acid.

15. The method of claim 11, wherein said composition further comprises carnitine.

16. The method of claim 11, wherein said composition further comprises creatine.

17. The method of claim 11, wherein said composition further comprises an element, wherein said element is selected from the group consisting of iron, zinc, manganese, magnesium, copper, cobalt, chrome, molybdenum, vanadium and selenium.

18. The method of claim 11, wherein said composition further comprises a vitamin wherein said vitamin is selected from the group consisting of Vitamin A, Vitamin B15, Vitamin C, Vitamin D3, and Vitamin E.

19. The method of claim 11, wherein said composition further comprises a bioflavonoid.

20. The method of claim 11, wherein said patient is human.
